SEQUENCE LISTING (13 sequences)

SEQ ID NO: 1 - DNA, 45 bp, Artificial Sequence
  Oligonucleotide primer
aactggaagaattcgcggccgcaggaatttttttttttttttttt

SEQ ID NO: 2 - DNA, 12 bp, Artificial Sequence
  Oligonucleotide primer
ggccgcaggaat

SEQ ID NO: 3 - DNA, 53 bp, Artificial Sequence
  Oligonucleotide primer
tgttaccaatctgaagtgggagcggccgcnnnnnntttttttttttttttttt

SEQ ID NO: 4 - DNA, 60 bp, Artificial Sequence
  Oligonucleotide primer
tgttaccaatctgaagtgggagcggccgcnnnnnnttttttttttttttttttttttttt

SEQ ID NO: 5 - DNA, 43 bp, Artificial Sequence
  Oligonucleotide primer
aactggaagaattaattaaagatcttttttttttttttttttt

SEQ ID NO: 6 - DNA, 296 bp, Artificial Sequence
  pT7T3-Pac polylinker
caccccaggctttacactttatgcttccggctcgtatgttgtgtggaattgtgagcggataacaatttcacacaggaaacagctatgacatgattacgaatttaatacgactcactatagggaatttggccctcgaggccaagaattcccgactacgtagtcggggatccgtcttaattaagcggccgcaagcttattccctttagtgagggttaattttagcttggcactggccgtcgttttacaacgtcgtgactgggaaaaccctggcgttacccaacttaatcgccttgcag

SEQ ID NO: 7 - DNA, 24 bp, Artificial Sequence
  Oligonucleotide primer
agcggataacaatttcacacagga

SEQ ID NO: 8 - DNA, 24 bp, Artificial Sequence
  Oligonucleotide primer
gactggtgagtactcaaccaagtc

SEQ ID NO: 9 - DNA, 20 bp, Artificial Sequence
  Oligonucleotide primer
taatacgactcactataggg

SEQ ID NO: 10 - DNA, 20 bp, Artificial Sequence
  Oligonucleotide primer
attaaccctcactaaaggga

SEQ ID NO: 11 - DNA, 54 bp, Artificial Sequence
  Oligonucleotide primer
cctcgtgccgaattcttggcctcgagggccaaattccctatagtgagtgtatta

SEQ ID NO: 12 - DNA, 56 bp, Artificial Sequence
  Oligonucleotide primer
attaaccctcactaaagggaataagcttgcggccgctttttttttttttttttttt

SEQ ID NO: 13 - DNA, 68 bp, Artificial Sequence
  Oligonucleotide primer
attaaccctcactaaagggaataagcttgcggccgcttaattaaagatcttttttttttttttttttt

(19) United States
(12) Patent Application Publication
(10) Pub. No.: US 2002/0172954 A1
(43) Pub. Date: Nov. 21, 2002

(54) Method for large scale cDNA cloning and sequencing by circulating subtraction

(76) Inventors: Yumin Mao, Shanghai (CN); Yi Xie, Shanghai (CN)

Correspondence Address: BAKER & BOTTS, 30 ROCKEFELLER PLAZA, NEW YORK, NY, 10112

(21) Appl. No.: 09/938,744

(22) Filed: Aug. 24, 2001

Related U.S. Application Data
(63) Continuation of application No. PCT/CN00/00036, filed on Feb. 25, 2000.

(30) Foreign Application Priority Data
Feb. 26, 1999 (CN) 99102450.8

(51) Int. Cl. C12Q 1/68; C12P 19/34

(52) U.S. Cl. 435/6; 435/91.2

(57) ABSTRACT

The present invention provides a method of cDNA sequencing by circulating subtraction, which comprises the steps of: (1) constructing the cDNA original libraries with high quality using the organism tissues as materials; (2) homogenizing the cDNA original libraries of step (1) according to the graded C₀t value, thereby obtaining the homogenized cDNA libraries corresponding to the graded C₀t value; (3) selecting and sequencing the clones from each of the homogenized cDNA libraries of the preceding step; (4) hybridizing and subtracting the homogenized cDNA libraries of the preceding step by using the sequenced cDNA clones; (5) repeating steps (3) and (4) 1-5,000 times. Further, a step of preliminary subtractive hybridization may be included in the method. By using said method, one can sequence cDNA of certain tissues or species efficiently and comprehensively.

The present invention is a continuation of PCT/CN/00036 filed Feb. 25, 2000, now published in Japanese as WO 00/52200, which claims the benefit of CN 99102450.8 filed Feb. 26, 1999.

### FIELD OF THE INVENTION

The present invention relates to the field of genetics, molecular biology and cDNA cloning. In particular, the present invention refers to a method of large-scale and comprehensive cDNA sequencing in any types of cells in one species.

### BACKGROUND OF INVENTION

With the development of biology, especially molecular biology, it is already well known that it is the genetic materials—genes that decide the biological characters. In order to understand all kinds of biochemical process deeply, to discover the relationships between diseases and genes, and to develop a crop with high quality, it is necessary for people to know the DNA information of an organism as completely as possible.

In any species, especially species of eukaryotes, the gene expressions have obvious differences from space to space and from time to time. Even for the same organism, the different types of cells will express different genes, and even the same cell in different period may express different genes. For example, the total genes in the human genome are estimated to be 50,000-100,000. (Bishop, J. O., J. G.Morton, M.Rosbash,and M.Richardson.(1974) Three abundance classes in Hela cell messenger RNA. Nature 250:199-204) In any given cell type, only about 10,000 genes are expressed and the average expression quantity of these genes in a single cell ranges from 200,000 copies to less than one copy. So, a great obstacle exists in the art to identify and sequence all or substantially all of the expressed genes.

With the improvement of PCR technology, it has been possible to prepare a cDNA library from a single cell. (Li, H., Gyllensten, U. B., Cui,X., Saiki, R. K., Erlich, H. A. & Arnheim, N.(1988) Nature (London) 335,414-417). Further, theoretically, it is possible to prepare a series of cDNA libraries that represent most or even all of the genes. First, preparing a cDNA library, then randomly selecting cDNA clones from the cDNA library and performing the large-scale and non-repeated sequencing, has been proved to be an efficient approach to discover new genes. (Adams, M. D., J. M. Kelley, J. D. Gocayne, M. Dubnick, M. H. Polemeropoulos, H. Xiao, C. R. Kerlavage, W. R. McCombie, and J. Craig Venter. (1991) Complementary DNA sequencing: Expressed sequence tags and Human Genome Project. Science 252:1651-1656; Adams, M. D., A. R. Kerlavage, C. Fields, and J. C. Venter.(1993a) 3,400 new expressed sequence tags identify diversity of transcripts in human brain. Nature Genet. 4:256-257; Adams, M. D., M. B. Soares, A. R. Kerlavage, C. Fields, and J. C. Venter. (1993b) Rapid cDNA sequencing (expressed sequence tags) from a directionally cloned human infant brain cDNA library. Nature Genet. 4:373-380.; Adams, M. D., A. R. Kerlavage, R. D. Fleischmann, R. A. Fuldner, C. J. Bult, N. H. Lee, E. F. Kirkness, K. C. Weinstock, J. D. Gocayne, O. White, et al.(1995) Initial assessment of human gene diversity and expression patterns based upon 83 millions nucleotides of cDNA sequence. Nature 377:3-174.; Khan, A. S., A. S. Wilcox, M. H. Polymeropoulos, J. A. Hopkins, T. J. Stevens, M. Robinson, A. K. Orpana,and J. M. Sikela.(1992) Single pass sequencing and physical and genetic mapping of human brain cDNAs. Nature Genet. 2:180-185.; McCombie, W. R., M. D. Adams, J. M. Kelley, M. G. FitzGerald, T. R. Utterback, M. Khan, M. Dubnick, A. R. Kerlavage, J. C. Venter, and C. Fields.(1992) Caenorhabditis elegans expressed sequence tags identify gene families and potential disease gene homologues. Nature Genet. 1:124-131.; Okubo, K., N. Hori, R. Matoba, T. Niiyama, A. Fukushima, Y. Kojima,and K. Matsubara. (1992) Large Scale cDNA sequencing for analysis of quantitative and qualitative aspects of gene expression. Nature Genet.2:173-179.) The improvement of the technology of large-scale random cDNA sequencing will accelerate the ongoing projects for finding out new genes of human and other species.

However, various technological difficulties in large-scale random cDNA sequencing must be overcome:

First, because transcript abundance varies from cell to cell and gene expression varies from gene to gene in each type of cell, in order to sequence all the expressed genes of one species, it is necessary to obtain mRNA from cells in different growing periods, thus making an intact cDNA library become extremely large. In a cDNA library of about 10⁷ clones, the mRNA from any tissues and even the rare mRNA may be repeatedly cloned, thus making the subtraction and the selection of the lowest genes become almost infeasible. To overcome these difficulties, one should first find out a desirable method to construct a cDNA library, because the ordinary cDNA library usually contains some highly abundant but unwanted clones (referred as “garbage” clones). (Adams, M. D., J. M. Kelley, J. D. Gocayne, M. Dubnick, M. H. Polemeropoulos, H. Xiao, C. R. Kerlavage, W. R. McCombie, and J. C. Venter.(1991) Complementary DNA sequencing: Expressed sequence tags and Human Genome Project. Science 252:1651-1656; Adams, M. D., M. Dubnick, A. R. Kerlavage, R. Moreno, J. M. Kelley, T. R. Utterback, J. W. Nagle, C. Fields, and J. C. Venter. (1992) Sequence identification of 2,375 human brain genes. Nature 355: 632-634.) These “garbage” clones include:

(1). clones merely having the mRNA poly A trails;

(2). clones only having the very short inserted cDNA fragments;

(3). clones only having the 3′ end of linker—dT)₁₈ primer (the primer used to form the first cDNA chain linked to the synthetic linker)

(4). fused-clones which are produced from the linkage of some different mRNA in the linking reaction and are usually the false cDNA clones.

The existence of these clones not only seriously hampers the efficiency of large-scale sequencing, but also destroys the integrity of the sequencing results to a large extent.

In addition, as a universal rule, the abundance of a cDNA clone in the cDNA library is always consistent with the abundance of the corresponding mRNA in the cell. The analysis of the annealing dynamics shows that, based on the abundance, mRNA in any typical body cells can be classified into the following three classes:

<1> high abundance mRNAs (These mRNAs are usually of only 10-15 types having highly repeated regions. The amount of these mRNAs is about 10-20% of the total mRNA.)

<2> moderate abundance mRNAs (These mRNAs are usually of 1000-2000 types having moderately repeated regions. The amount of these mRNAs is about 40-45% of the total mRNA.)

<3> low abundance mRNAs, i.e., complicated mRNAs (These mRNAs with low copy number usually are of about 15,000-20,000 types. The amount of these mRNAs is about 40-45% of the total mRNA.) (Bishop, J. O., J. G. Morton, M. Rosbash, and M. Richardson. (1974) Three abundance classes in Hela cell messenger RNA. Nature 250:199-204.; Davidson, E. H. and R. J. Britten.(1979) Regulation of gene expression: Possible role of repetitives sequences. Science 204:1052-1059.)

Therefore, if one takes a new clone out of the library after the majority of mRNAs of Classes <1> and <2> have been identified, the possibility that said clone is from one of the sequenced sequences is over 60%. Thus, it is inevitable to carry out plenty of duplicate sequencing to find a new gene. The great difference of mRNA abundance results in plenty of duplicate and redundant sequencing. Even many of the high quality cDNA libraries commercially available are not the ideal material for large-scale cDNA sequencing.

In order to overcome the above difficulties, construction of high quality cDNA libraries, by homogenization and subtraction of said libraries is a promising new technology.

One of the homogenizing methods is the saturation hybridization of a DNA genome. (Weissman, S. M.(1978) Mol.Biol.Med. 4,133-143.) Although this method produces a homogenized cDNA library, but it is unfeasible in practice because it is difficult to provide a saturating quantity of rare cDNA to participate in the hybridization. Another method is to utilize annealing dynamics. Assuming second order kinetics, rarer species anneal much more slowly than the high abundance cDNAs. Thus, single-chain cDNA fragments after annealing will be gradually homogenized during the reaction. That is, the lower abundant cDNA is more likely to be retained, thereby causing the number of the clones with different abundance closer. (Weissman, S. M.(1987) Mol. Biol Med. 4,133-143.; Ko, M. S. H.(1990) Nucleic Acids Res.18, 5705-5711.; Patanjali, S. R., Parimoo, S. & Weissman, S. M.(1991) Proc. Natl. Acad. Sci. USA 88.1943-1947.) The method of homogenization maintains the emergence frequency of every clone in the cDNA library within a small range. According to the practical experience of some scholars, it is impossible to obtain a cDNA library having an identical mole of various cDNA.

Homogenization has the following advantages: (1) A homogenized cDNA library can accelerate the mapping of the clones so as to obtain the disease-associated genes, to improve the efficiency of the subtractive hybridization, to expedite the research on the arrangement of the expressed sequence tag or EST on the chromosomes and their locations on the cloned large DNA fragments from the genome (i.e., extron mapping). (2) By reducing the number of the identical clones, homogenization makes it possible to overlay the cDNA library onto a film in high density. (It is impossible to overlay onto a film 10⁷ clones from an unhomogenized library.) (3) Using random cDNA sequencing, homogenization can greatly accelerate the development of an EST database by increasing the emergence frequency of rare clones and decreasing the proportion of high abundance cDNA.

Selecting the homogenized library, i.e., the library in which the abundances of all clones are essentially same, is beneficial to large-scale sequencing. (Soares, M. B. 1994.Construction of directionally cloned cDNA libraries in phagemid vectors. In Automated DNA sequencing and analysis(ed. M. D. Adams, C. Fields and J. C. Venter),pp.110-114. Academic Press, New York, N.Y., Soares, M. B., M. F. Bonaldo, P. Jelenc, L. Lawton, and A. Efstratiadis. 1994. Consruction and characterization of a normalized cDNA library. Proc.Natl.Acad.Sci. 91:9228-9232.) This has been proved by the experiment finished by Berry etc. (Berry,R., T. J. Stevens, N. A. Walter, A. S. Wilcox, T. Rubano, J. A. Hopkins, J. Weber, R. Goold, M. B. Soares, and J. M. Sikela. 1995. Gene-based sequence-tagged-sites(STSs)as the basis for a human gene map. Nature Genet. 10:415-423.; Houlgatte, R., R. Mariage-Samson, S. Duprat, A. Tessier, S. Bentolila, B. Lamy, and C. Auffray. 1995. The Genexpress Index: A resource for gene discovery and genic map of human genome. Genome Res. 5:272-304.) Calculation results show that when the C₀t is 5.5, wherein C₀ represents total DNA concentration whose unit count as nucleotide for mol per liter; t represents time whose unit is second, the content of the high abundance mRNA of the three kinds of mRNA is sharply decreased and the abundances of various clones are restricted in a certain range. (Soares, M. B. 1994.Construction of directionally cloned cDNA libraries in phagemid vectors. In Automated DNA sequencing and analysis (ed. M. D. Adams, C. Fields and J. C. Venter), pp.110-114. Academic Press, New York, N.Y.)

Because a relatively large portion of the human genes have been identified, it is impossible to completely rely on the method of library homogenization so as to avoid duplicate sequencing those genes expressed in several tissues. Therefore, Soares deems that it will bring more advantage in the full-scale sequencing by using the subtractive cDNA libraries that enrich the low expressed genes and the unidentified genes.

In attempts to improve the representation of the longest cDNA clones in the library, Soares et al. established four methods for constructing the homogenized library and successfully constructed various homogenized libraries including fifteen human libraries, three mice libraries, two rat libraries and one Schistosoma mansoni library. The human libraries and the mice libraries were provided to the IMAGE (an organization for molecular analysis of human genome and expressing). (Lennon, G. G., C. Auffray, M. Polymeropoulos, and M. B. Soares.1996. The I.M.A.G.E. Consortium: An integrated molecular analysis of genomes and their expression. Genomics 33:151-152.). A total of 315,408 expressed sequence tags (ESTs) were obtained from these libraries. The above reference gives a detailed description and a comparison of these four methods for homogenizing cDNA libraries. Furthermore, a simple scheme for constructing the subtractive cDNA library is also described in detail in this reference.

Although there are some methods of homogenization and subtraction, there are still many disadvantages. In the published articles of Soares et al. in 1994, one method of homogenization, referred hereinafter as “Soares Method 1”, was described in detail. (Soares, M. B. 1994. Construction of directionally cloned cDNA libraries in phagemid vectors. In Automated DNA sequencing and analysis(ed. M. D. Adams, C. Fields and J. C. Venter),pp.110-114. Academic Press, New York, N.Y.) Said method was used to construct the 1NIB and 1NFLS homogenized libraries, from which 45,192 and 86,088 ESTs were obtained, respectively. Using the method of homogenization, the abundance of clones can be limited to a narrow range. However, the comprehensive identification of these two libraries by Southern analysis shown that the truncated clones are more easily reserved than the corresponding intact clones during the homogenization process of Soares Method 1.

As to why truncated clones are more easily reserved than the corresponding untruncated clones during the homogenization process of the Soares Method 1. (Soares, M. B. 1994. Construction of directionally cloned cDNA libraries in phagemid vectors. In Automated DNA sequencing and analysis (ed. M. D. Adams, C. Fields and J. C. Venter),pp.110-114. Academic Press, New York, N.Y.). One should first refer to Soares Method 1 which mainly comprises the following steps.

(1) annealing the single-stranded DNA (ssDNA) prepared from a cloned cDNA library with the oligo(dT)₁₈ primers;

(2) in the presence of deoxy- and dideoxy-NTPs, performing the primer extending reaction to produce the 340 extension products of the non-coding chain with a length of 200-300 bp;

(3) purifying a portion of the double-stranded circular DNA produced in the above step by HAP chromatography;

(4) denaturing the double-stranded circular DNA obtained in Step (3), and renaturing them in a relatively low C₀t (C₀t is about 5-10);

(5) purifying the remaining single-stranded circular DNA by HAP to obtain the homogenized library;

(6). converting the single-stranded circular DNA into the double-stranded circular DNA;

(7). introducing the double-stranded plasmid into the host cell by electroporation.

Because the probability for producing the truncated cDNA is lower than that for producing the corresponding untruncated cDNA, in the reaction, the cDNA amplification product is easier to anneal with an intact cDNA having an overlapping region than the template itself (i.e., the truncated cDNA). On the other hand, the amplification products of the untruncated cDNA are most likely to be annealed with their templates, and not with the untruncated cDNA, because the amount of the untruncated cDNA is larger than the truncated cDNA and the possibility that an overlapping region exists between the extension fragments produced by the untruncated clones and the truncated clones is very low. The result is that the untruncated single-stranded clones may renature with more than one extension product which are not overlapped with each other, while the truncated clones remain single-stranded so as to be retained in the elution solution after purifying by HAP, thereby forming a part of the homogenized library.

In order to solve these problems, Soares designed Methods 2-1 and 2-2, whose processes are almost the same except the hybridization conditions. Using these methods, one can use the plasmid DNA prepared from the original library as a template to synthesize RNA. Said RNA fragments are then used as the drivers to hybridize with the same library of single-stranded circular DNA. These two improved methods increase the expressivity of the longest cDNA clones in the homogenized library.

However, in the libraries constructed by Methods 2-1 and 2-2, Soares found some cDNA clones, e.g., the alpha- lactoprotein gene in the 5Nb2HFLS20W liver/spleen library and the G3PD gene in the breast library, which are much more difficult than any other clones to be homogenized. Although this problem was somewhat solved in Method 2-3, the degree of homogenization thereof can not reach the same level of homogenization of Method 1. Further, Method 2-3 is not a real homogenization process, because the purpose of this method is not to make the abundances of all the cDNA clones to be the same or similar but to reduce the expressivity of clones with the highest abundance.

Soares, based on the combination of Methods 1 and 2, attempted to establish Methods 3 and 4 so as to achieve not only the sufficient homogenization as in Soares Method 1, but also the high expressivity of the longest cDNA clones as in Methods 2-1, 2-2 and 2-3. However, this is only a partial improvement and the effect is not significant.

So it is an urgent need in the art to develop a technical solution for efficient, complete and large-scale sequencing of cDNA so as to powerfully accelerate the development of molecular biology, genetics, medication, breeding, etc.

The purpose of this invention is to provide a method suitable for sequencing cDNA effectively and extensively. This method is suitable for large-scale sequencing of cDNA in various eukaryotes, especial the higher plants and animals, e.g., the mammals.

### SUMMARY OF THE INVENTION

The purpose of this invention can be achieved by the following embodiment of the invention. A method of sequencing cDNA which comprises the steps of:

(1) constructing cDNA original libraries in which the length of the inserted fragments is 0.5-3.0 kb, by using tissues of organisms as materials;

(2) homogenizing the cDNA original libraries of step (1) according to the graded C₀t value, thereby obtaining the homogenized cDNA libraries corresponding to the graded C₀t, wherein C₀ is concentration of the total DNA (in mol/L) based on the number of nucleic acids; and t is the renaturing time (in seconds);

(3) selecting and sequencing 5-500 clones from each of the homogenized cDNA libraries of the preceding step;

(4) synthesizing probes corresponding to the sequenced sequences by using the sequenced cDNA clones, and hybridizing and subtracting the homogenized cDNA libraries of the preceding step with said probes, thereby removing the sequenced cDNA clones from the cDNA libraries and forming the subtracted homogenized cDNA libraries; (5) repeating Steps (3) and (4) 1-5,000 times.

### BRIEF DESCRIPTION OF THE DRAWINGS

FIG. 1: Schematic diagram of a process of homogenizing under the graded C₀t values.

FIG. 2: Schematic diagram of a process of pre-subtraction by hybridization.

FIG. 3: Schematic diagram of circulating subtraction.

### DETAILED DESCRIPTION OF THE INVENTION

The present invention provides a method to efficiently and completely sequence the genes expressed in an organism, a tissue, or a cell. This method includes the following steps: constructing a high-quality cDNA original library to ensure that the clones in the library correspond to all of or almost all of mRNA; dividing the C₀t value into different grade so that the cDNA of different abundance can be reserved in one or more homogenized cDNA libraries; removing the sequenced cDNA clone from the homogenized cDNA libraries by hybridization and subtraction. Therefore, after hybridization and subtraction, the homogenized cDNA libraries only contain the unsequenced cDNA clones, thereby significantly improving the efficiency of the sequencing in the next cycle.

In order to study gene expression in different tissues, the method of the invention comprises: using different tissues of the organism as materials to construct cDNA original libraries of different tissues; homogenizing said cDNA original libraries respectively to obtain homogenized libraries of different tissues; hybridizing and subtracting among said homogenized cDNA libraries of different tissues, thereby forming the homogenized cDNA libraries subtracted by hybridization. Further, hybridization and subtraction between cDNA original libraries of different tissues can be done so as to obtain a cDNA original library which contains differentially expressed cDNA.

In this invention, “different tissue” means any different tissues due to different organisms, different types, different phases of development and/or different status of pathology. The examples of different tissues include, but are not be limited to, the tissues of different types in an organism, such as human brain and muscle; the tissues of different phase of development in an organism, such as brain in the embryonic and adult stage; normal tissues and pathologic tissues in an organism, such as a normal brain and a brain of senile dementia; and tissues of different organisms, such as the brain of rats and rabbits, etc.

For the constructed cDNA library to be sequenced, one may first hybridize and subtract it with a cDNA original library of different tissues or a homogenized cDNA library so as to sequence the cDNA expressed differentially. In particular, a cDNA library of pathologic tissues can be hybridized and subtracted with a cDNA library of normal tissue so as to obtain the cDNA expressed differentially in the pathologic tissue and sequence it. Similarly, the cDNA clones containing genes expressed differentially in the different phases of development can be obtained by hybridizing and subtracting between the cDNA libraries of different phases of development of the same tissue.

Furthermore, in order to improve the efficiency of sequencing and reduce duplicate sequencing, one may synthesize artificial probes in the steps of hybridizing and subtracting based on the information of the known cDNA sequences, especially for the organism, e.g., human, whose cDNA sequences are relatively well known, or based on other sequenced clone. Said probes are then used to hybridize and subtract the cDNA original library or the homogenized cDNA library.

The sequencing method used in the invention can be any of the conventional method in the art, manually or automatically. A useful procedure of DNA sequencing comprises the steps of:

for the sequenced fragments, determining whether they are new cDNA clones;

for the new clones, analyzing the integrity of 5′ end;

for the new clones having an intact 5′ end, sequencing continuously until obtaining the full-length sequences.

In the invention, the C₀t value is typically divided into 3-8 grades in the process of homogenization. However, it is also possible to divide it into more grades, such as 10 grades or less grades, e.g., 2 grades. Preferably, C₀t is divided into 3-5 grades.

In one embodiment, the C₀t is divided into 3 grades as follows: 0<C₀t<1, C₀t=1-50, and C₀t>50, thereby, from the same cDNA original library, obtaining the homogenized libraries selecting from the group consisting of (a) highly repeated homogenized cDNA libraries, (b) moderate repeated homogenized cDNA libraries, and (c) lowly repeated homogenized cDNA libraries. In another embodiment, the C₀t is divided into 4 grades as follows: 0<C₀t<0.5; C₀t=0.5-10; C₀t=10-50 and C₀t>50. Especially for sequencing the low abundance cDNA, the C₀t is preferred to be divided into more grades in the range of 0-5.

One key for successful sequencing is obtaining a high quality cDNA original library that contains most or all of the expressed genes, and few “garbage” clones. Therefore, an appropriate amplification technique of high efficiency should be used to amplify the extracted mRNA to form the corresponding cDNA. The examples of the technique include, but are not be limit to, mixed reverse transcriptase technique, SMART PCR technique, nucleotide capping technique and the combination thereof. Furthermore, it is necessary to separate the amplified fragments and remove the short fragments (e.g., less than 200 bp). Then, the cDNA original library can be constructed by inserting the fragments whose lengths meet the requirement into the appropriate vector.

A method useful for constructing the cDNA original library in the invention comprises the following steps:

extracting mRNA, amplifying mRNA to obtain the corresponding cDNA by a technique selected from the group consisting of: mixed reverse transcriptase technique, SMART PCR technique, nucleotide capping technique and the combination thereof;

separating and collecting cDNA fragments of 0.5-3.0 kb;

cloning the separated cDNA fragments into suitable vectors;

separating the vectors comprising the inserted fragments of 0.5-3.0 kb;

transforming into suitable bacteria, thereby obtaining the cDNA original libraries.

Further, the separation and collection of cDNA fragments of 0.5-3.0 kb can be carried out by electrophoresis and cutting gel, or by gel chromatography purification; and the separation of the vectors comprising the inserted fragments of 0.5-3.0 kb can be carried out by reversed phase HPLC.

Another important key to this invention is that a series of technical steps for constructing high quality cDNA original library are provided. The purpose of these steps is to reduce the number of garbage clones included into the cDNA original library from the very beginning and to the largest extent. The most intact cDNA is amplified by the highly efficient nucleic acid amplification techniques, and the clones are purified prior to or right after cloning into the vectors. These technical steps ensure the cDNA in the cDNA original library is long enough.

Furthermore, Scoares, et al (Proc.Natl.Acad.Sci.USA Vol.91, pp.9228-9232) reported that about 13% of the clones in the IB library (from human mRNA) lack the poly (A) tail. It was proposed that abnormal priming during the amplification may cause this. Therefore, in order to further improve the quality of cDNA original library, a treatment with a d(T) 18-25 affinity column can be used to greatly decrease the amount of the clones that lack the poly (A) tail, thereby constructing a better cDNA original library.

In order to determine various genes in an organism and ensure a smooth and efficient large-scale random cDNA sequencing, one desirable method is to construct enough or all of the high quality cDNA original libraries from one species (such as human). For example, these cDNA original libraries can be from human, mouse, rats or schistosome. These cDNA original libraries can also be from the normal tissues or typical pathological tissues of an organism, or from the tissues in the various development phase. The materials used to construct the cDNA original library are various tissues. The examples include, but are not be limited to, (a) brain of human infant, liver and spleen of human embryo, human parturient placenta, human placenta aged 8-9 weeks, human breast, adult brain, human retina, human pineal gland, human ovary tumor, human melanin cell, the heart of human embryo, human parathyroid gland tumor, human senile fibroblast, human sclerosis focus, lung of human embryo, (b) 19.5 day old mouse embryo after amphimixis, 17.5 day old mouse embryo after amphimixis, 13.5-14.5 day old mouse embryo after amphimixis, (c) heart and spleen of rat, (d) adult schistosome of 8 week and so on.

The reason why it is necessary to obtain enough high quality cDNA original libraries is because, in a single cell, about ⅓ of the expressed mRNAs have 1-10 copies (Galau, G. A., Britten, R. J.&Davidson, E. H.(1977) Proc. Natl.Acad.Sci.USA 74,1020-1023.). A single cell can express 10,000 mRNA or more. If each mRNA produces 2 or 3 cDNA in a length of 500-2000 bp, then the expressivity of each cDNA is 1/30,000 after homogenization. The expressivity of individual cDNA is lower if the cDNA is from complex tissues. The expressivity of each fragment is lower than 1/100,000 if the library originates from all of the mRNA expressed in different phases.

Therefore, the fully representative library, in which the expressed sequences of various cells are included, can be obtained by the following steps: the libraries are constructed from a proper amount of organs or tissues, pre-homogenized; and then each library is homogenized specially.

Secondly, another important key of the invention is that we proposed to homogenize the high quality library under enough and different C₀t, based on sufficient high quality cDNA original libraries. Therefore, all of the cDNA from a normal tissue or typical pathologic tissue can be distributed in the various homogenized sublibraries. Each sublibrary represents a set of cDNA whose homogenization is higher and more specific. The total of these sublibraries prepared from a cDNA original library of a normal or pathologic tissue correspond to the total cDNA of the normal or typical pathologic tissue.

Furthermore, manifold homogenized sublibraries, which only correspond to the normal tissue or typical pathologic tissue, can be obtained by subtraction. The subtraction includes the step of using the probes, which are prepared from the sequenced cDNA clones, to hybridize with the cDNA original library or sublibraries to remove the sequenced cDNA clone.

In the art, the artisans usually try to find several optimal C₀t so as to solve the question without success. They deem hybridization as a simple secondary dynamics reaction, and anticipate that the expressivity of the most abundant component in single-stranded DNA is less than twice the least abundant component, if 50% of the least abundant component are renatured in the mixture. In fact, hybridization is much more complex than anticipated. Hybridization of the most abundant component is faster than anticipated. In the early studies, expressivity of the most abundant component in single-stranded DNA was even lower than that of the least abundant component. The main reason for this effect is that double-stranded fragments probably continue to hybridize with the remaining single-stranded cDNA. So the solution in the art is to select appropriate timing of renaturation and concentration of the renaturation reactant so that the effect can be reduced to an acceptable level. Furthermore, adding hydroxyapatite in the various phase of reaction to remove the double-stranded products can further reduce the effect. But hybridization and subtraction with only a few of C₀t values can not fully solve the problem and the results are not satisfied.

Moreover, in Smith, M. J., Brititen, R. J. & Davidson, E. H. (1975)Proc.Natl.Acad.Sci. USA72, 4805-48 09.; Marrow, J. F.(1974) Doctoral thesis (Stanford Univ., Standford, Calif.), PP,101-190. Mol.Cell.Biol.10,243-253, the experimental results did not conform with the prediction based on the secondary dynamics law. First, the most abundant cDNA decreases more quickly than predicted, and the number thereof in the single-strand mixture is less than that of some low abundance cDNAs. Second, even if during the shortest renaturation time, considerable rare cDNAs are found in the double-stranded mixture (Both are inconsistent with the prediction). The explanation for the first abnormal phenomenon is described in this literature (Smith, M. J., Britien, R. J. & Davidson, E. H. (1975) Proc.Natal.Acad.Sci.USA 72,4805-4809.; Marrow, J. F. (1974)Doctoral thesis (Stanford Unive.,Stanford,Calif.), PP, 101-190. Mol.Cell.Biol.10,243-253.). During the process of renaturation, if there is a homologous region, long fragments and short fragments from the same kind of mRNA will hybridize and produce a single-stranded tail. Thus, this structure can pair with the homology fragment in the remaining single-stranded DNA and does not follow the secondary dynamics reaction law. This dynamics action causes the additional convert from single strand to double strand. So, even if the very low abundance mRNAs, such as c-myc and TCR, may exist in the mixture of single-stranded components and the mixture of double-stranded components during any renaturation time.

Soares, et al (Proc.Natl.Acad.Sci.USA Vol.91,pp.9228-9232) constructed two human fetal brain libraries (¹NIB library and ²NIB library) under two C₀t values of 5.5 and 2.5. It is found that the frequency of occurrence of some clones decrease in the ¹NIB library but increase in the ²NIB library.

For example, based on the calculation of estimated mRNA abundance in brain, the optimal degree of enrichment of mRNA is achieved when C₀t is 5-10. However, this is only suitable for relatively abundant cDNAs. If C₀t is too low (1), only the most abundant mRNAs (Class <1>) are enriched; moderate abundance mRNAs (Class <2>) are unable to be enriched. On the other hand, if C₀t is too high (50), the degree of enrichment of the most abundant mRNA is insignificant while expressivity of the most complicated mRNA (Class <3>) becomes high. So, it is proposed in the invention that, for a cDNA original library, all of cDNAs can not be distributed into the different homogenized libraries unless different C₀t values are selected to construct a series of homogenized libraries, depending on the different organism sources and/or sequencing purposes.

As the third important key of the invention is the circulating subtraction of the homogenized library after every selected clone from the homogenized library is sequenced. Thus, the sequenced clones are removed and the unsequenced clones are retained to be sequenced in the next cycle. Therefore, the sequencing efficiency is improved significantly and the possibility for duplicate sequencing is decreased dramatically.

In particular, the method of the invention includes the following steps:

(1) Constructing a High Quality cDNA Original Library

For the conventional methods of constructing cDNA library, see the above description. Preferably, in this present invention, the following one or more of techniques are used: mixed reverse transcriptase technique (Usage FD heat-resistant reverse transcriptase; Fudan transaction 37(2):225-228; China Biochemistry And Molecular Biochemistry 14(2):170-174,1998); SMART PCR technique (Clontech, SMART™ PCR cDNA Library Construction Kit User Manual (PT3000-1); (Kazuo Maruyama and Sumio Sugano(1994) Oligo-capping: a simple methods to replace the cap structure of eukaryotic mRNAs with oligoribonucleotides. Gene 138, 171-174. Caminci, P. et al. (1996) High-efficiency full-length cDNA cloning by biotinylated CAP trapper. Genomics 37,327-336.). Thus, the ratio of full-length cDNA in the whole products is increased. In order to assure the ratio of full-length cDNA in the whole cDNA original library, it is preferable to isolate the amplified cDNA fragments with the desired length (usually at least 200 bp) after amplification, and clone them into appropriate vectors. Finally, the resultant vectors are loaded onto the HPLC reverse phase column and the peaks containing the vectors that contain inserted fragments (e.g., 0.5, 3.0.2 kb) are collected. The collected vectors are transformed into the appropriate bacteria, thereby obtaining a high quality cDNA original library.

Using the above steps, the ratio of garbage clones in the high quality cDNA original library is so small that it can be neglected.

One desirable strategy is to get the high quality cDNA original libraries of different nonnal tissues or typical pathologic tissue of an organism as many as possible so as to obtain the cDNA original library which overlays all of or almost all of expressed genes of the organism.

(2) Homogenizing Under Graded C₀t Values

Then, using the methods of homogenization in the art, the high quality cDNA original libraries of the preceding step are homogenized. The difference is to divide C₀t into several grades and to homogenize under different C₀t values. Because there is no best C₀t value under which one can not only achieve the homogenization but also retain all kinds of cDNA clones corresponding to different abundences. On the contrary, only if a series of homogenized libraries are constructed under different C₀t values, can all of cDNAs be distributed into different homogenized libraries.

In particular, depending on the cDNA original libraries from different origins, one can choose enough different C₀t values, wherein C₀ represents total DNA concentration whose unit is mole of the nucleic acid per liter and t represents time whose unit is second(s).

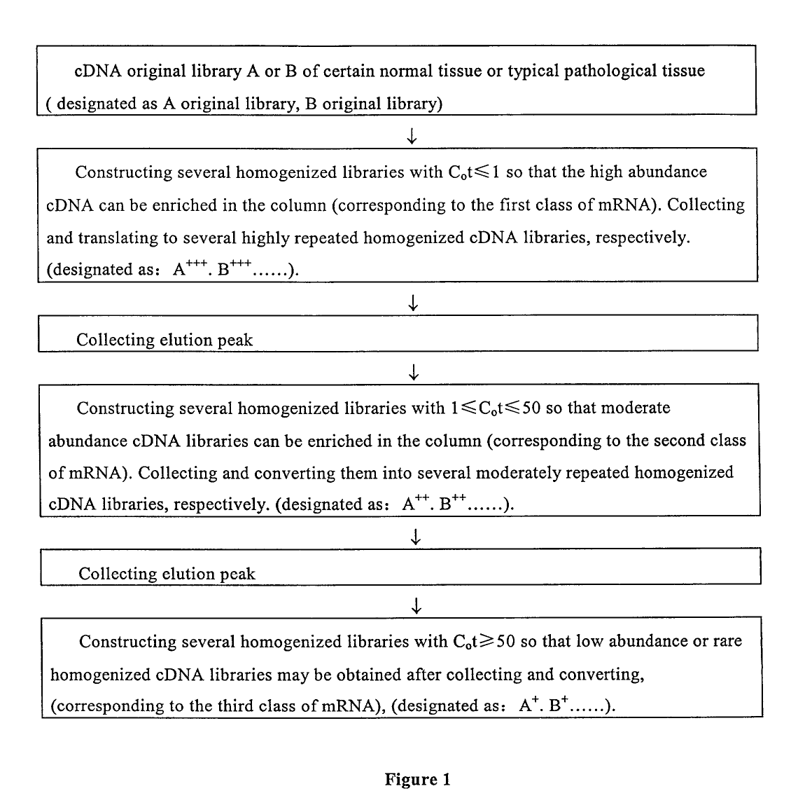

For example, the C₀t is divided into three grades such as high, moderate, and low grades, and several different C₀t values are selected in each grade. Therefore, one cDNA original library will be divided into three classes of homogenized libraries, i.e., high abundance homogenized cDNA libraries (C₀t 1), moderate abundance homogenized cDNA libraries (C₀t=1-50), and low abundance homogenized cDNA libraries (C₀t 50). Each class of homogenized libraries can be further divided into more homogenized sublibraries. The process is shown in FIG. 1.

(3) Pre-subtraction by Hybridization

The process of pre-subtraction by hybridization is optional. In order to find target genes directly or quickly, the homogenized libraries may be pre-subtracted by hybridization. For example, pre-subtraction by hybridization can be performed among the homogenized cDNA libraries of the same abundance (such as high abundance) from different tissues, such as brain and liver, so as to rapidly determine the cDNA expressed differentially. Alternatively, pre-subtraction by hybridization can be performed between the cDNA libraries of normal and pathologic tissues of the same tissue (such as brain tissue). Further, pre-subtraction by hybridization can be performed among the homogenized cDNA libraries of different abundances of the same normal tissue (such as high and low abundance homogenized cDNA libraries) so as to focus on the sequencing of low abundance cDNA.

Therefore, on the basis of existing homogenized methods, subtractive homogenized libraries may be obtained after pre-subtraction by homogenization among different cDNA libraries.

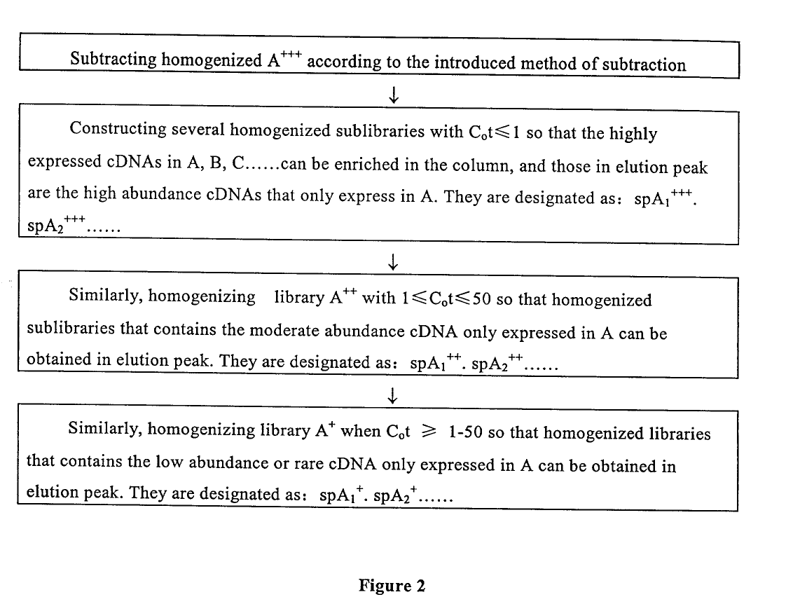

The process is shown in FIG. 2, wherein non-A cDNA original library (including B original library, C original library, etc.) are used as templates to synthesize DNA or RNA fragments which are used as drivers. The drivers are the single-stranded DNA or RNA fragments hybridizing with the original library.

(4) Selecting Clones for Sequencing

According to the conventional sequencing method in the art, 5-500 clones are selected randomly from the homogenized cDNA libraries, which have been treated with or without pre-subtraction by hybridization, of the preceding step, judged whether they are the new cDNA clones. If so, integrity analysis of 5′ end is carried out until obtaining full-length sequences.

(5) Subtractive Hybridization

The probes are synthesized on the basis of sequenced cDNA libraries consisting of all the long sequenced cDNA clones, and hybridized with the cDNA original libraries, or the homogenized libraries or the subtracted homogenized libraries obtained from the previous step. Thus, the cDNA clones that have been sequenced in the previous step are removed from the libraries and the unsequenced clones are retained. The efficiency of large-scale sequencing is therefore remarkably increased. The retained unsequenced clones make up the subtractive homogenized cDNA libraries, which are used in the next cycle.

(6) Repeating Step (4) of Sequencing and Step (5) of Subtractive Hybridization

By repeating the sequencing (4) and step (5) of subtractive hybridization, new sequences are sequenced efficiently and the unsequenced cDNA is reserved for the next cycle. There is no limit for the number of repeating or cycle, usually 1-5000 times. The number depends on the circumstances and the need, for example, on the basis of whether the desired genes have been obtained, or on the amount of clones in each cycle.

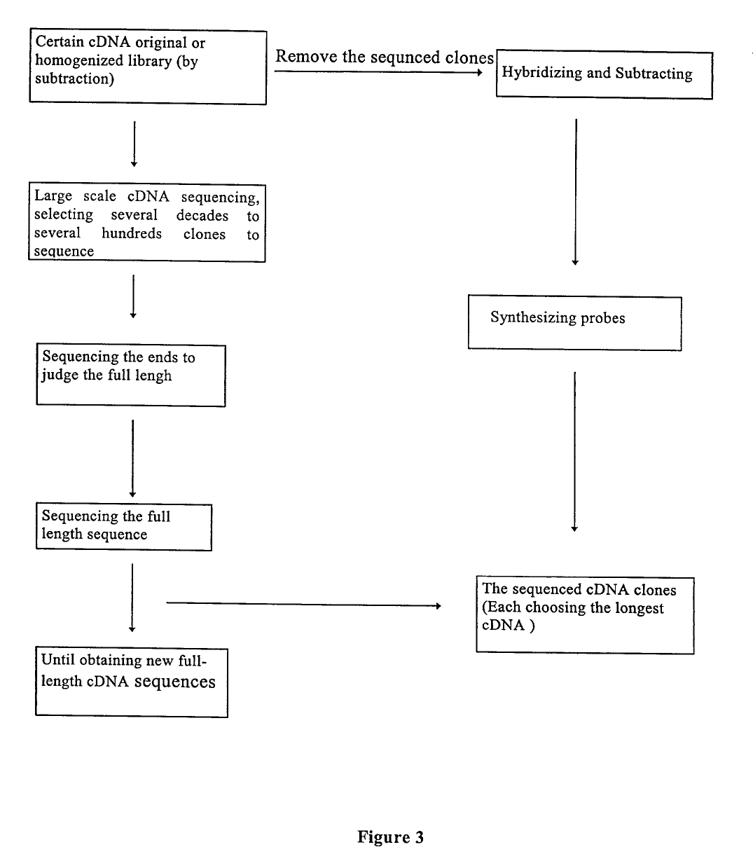

This process is shown in FIG. 3.

The invention is further illustrated by the following non-limiting examples.

### EXAMPLES

### General Methods

(A) HPLC Reversed Phase Chromatography

Reversed phase chromatography (RPC) has high resolution. Because the material can tolerate high pressure, it can be used in HPLC column and make isolation efficiently and rapidly. As a result, it becomes more and more widespread in the isolation and purification of nucleic acid. It also has the following advantages:

(1) Isolating oligonucleotide of different sizes.

The resolution is high, especially in isolating copolymerized adenine nucleotide of 10-130 bp. It can isolate fragments differing by only one nucleotide. The recovery ratio is high and the purity of the recovered sample is good.

(2) Isolating fragments of DNA restriction endonuclease.

It can isolate fragments differing only by 4 bp.

(3) Isolating and purifying closed circle (“cc”) plasmid DNA molecular.

When isolating the crude products of plasmid DNA on RPC column, the purified cc molecular can be obtained.

Moreover, HPLC can also be used to isolate and purify plasmids, remove contaminating protein and other nucleic acids (such as RNA of high molecular weight), and efficiently isolate plasmids differing by 100-200 bp. The typical operating conditions are as follows:

Column: Bio-Gel plasmid column

Size: 50×7.8 mm

Cetolog number: 125-0537

Sample: PUC₁₈ 100 ug

Mobile phase:

A:0.02M potassium phosphate, 50% formamide, pH6.7

B:0.02M potassium phosphate, 50% formamide, pH6.7, 1.5M KCl

Gradient: 50% B 10 min, 50-70% B 30 min

Flow rate: 1.0 ml/min

Detection: 1 uv@270 nm

(B) Hydroxyapatite Column Chromatography

The method of isolating single-stranded DNA and double-stranded DNA with hydroxyapatite has been mentioned in details in the literature (Britten, R. J., Graham, D. E. & Neufeld, B. R. (1974) Methods Enzymol. 29, 363-418.). The concentrations of sodium dihydrogen phosphate used for eluting DNA single-stranded components and double-stranded components were 0.1M and 0.35M, respectively. However, as the single-stranded components of different sizes obtained from HaeIII-digested M13mp13 DNA, the double-stranded DNA of less than 200 bp would also be eluted by 0.1M phosphate buffer. In order to avoid contamination of the single-stranded DNA by this kind of tiny double-stranded DNA fragments, all the DNA components used in process of homogenization were over 400 bp.

Hydroxyapatite (DNA-grade Bio-Gel HTP) produced by Bio-Rad Inc. was suspended in 0.01M sodium dihydrogen phosphate (pH7.0) containing 0.1% SDS in the proportion of 1:10. The homogenate was then poured into chromatography column (volume, 1.0 ml) containing a water jacket, the flow rate of eluent was 8-10 ml/hr. Its binding and eluting characters were tested by HaeIII digestion products of M13mp18 single-stranded DNA and 1 kb double-stranded DNA sequence ladder (Life Technologies Inc.) as the marker for double-stranded DNA. The temperature of the chromatography column was maintained at 60° C. by circulative hydrographic basin produced by Neslab Inc. The calibration of the conditions of single-stranded and double-stranded DNA elution was completed by a graded series of sodium dihydrogen phosphate buffer, where the concentrations were from 0.05M to 0.4M and increased at an average rate of 0.02M. All the buffers contained 0.1% SDS.

(C) Desalination and Concentration of the Elution Peak

The single-stranded DNA eluted from the hydroxyapatite column was not suitable for PCR amplification. This is because: (i) sodium dihydrogen phosphate and SDS of high concentration existed, inhibiting the activity of Taq DNA polymerase; and (ii) the concentration of the single-stranded DNA in the eluent was too low. The following methods could be used to solve the problem: filtration with Centricon-30, purification with Glassmilk (according to the handbook provided by manufacturer), concentration with n-butanol and iso-butanol, dialysis after lyophilization, etc. Among these, the method of filtration with Centricon-30 was chosen to recover the single-stranded DNA (Stafford, D. W. & Bieber, D. (1975) Biochem. Biophys. Acta. 378, 18-21). Because the recovery ratio of this method reached 90% and it could remove the salt sufficiently.

The chromatography column eluent was centrifuged, applied to a Centricon-30 filter membrane so as to concentrate the volume of the eluent from 10-12 ml to 100-200 ul, and then washed with sterilized water and TE buffer (10 mM Tris. HCl, pH8.0/1 mM EDTA) at least three times.

### EXAMPLE 1

Construction of high quality cDNA original libraries from human normal tissue or typical pathologic tissue and homogenized and subtracted cDNA sublibraries only expressed in healthy infant brain, and the large scale cDNA sequencing.

The materials included in this example for construction of cDNA original libraries were as follows (As sequencing went on or according to requirements, cDNA original libraries from other normal tissue or typical pathologic tissue could be added):

Human fetal brain, human embryonic liver and spleen, human full-term placenta, human 8-9 weeks' placenta, human breast, adult brain, human retina, human conarium, human oophoroma, human melanocyte, human embryonic heart, human parathyroid cancer, human senile fibroblast, human poly-sclerotic focus, human embryonic lung.

In detail, the materials were:

(1) human fetal brain, from a 72-day female fetus who died of spinal amyotrophy;

(2) human liver and spleen, from a healthy 20-week female fetus;

(3) all the cell poly(A)+ mRNA, from healthy breast tissue obtained in breast plastic operation;

(4) all the adult brain RNA, from a 55-year old man who died of aortic aneurysm break, brain tissue (including right and left cerebrum, parietal bone, temporal bone, occipital cortex infracortical white matter, basal ganglion, thalamencephalon, mesencephalon, bridge, caudex dorsalls), obtained 17-18 hours after death;

(5) all the human healthy retina cell RNA, from a 55-year old Caucasian male;

(6) human healthy conarium, from three different races: (i) a 48-year old Caucasian male; (ii) a 18-year old Caucasian female; (iii) a 20-year old Negro American;

(7) all the human oophoroma cell mRNA, from a 36-year old third-stage mastoid cystadenocarcinoma patient, surface diffusion and metastasis having occurred;

(8) all the human melanocyte DNA, from healthy foreskin;

(9) healthy fetal heart and lung, from samples of a 19-week fetal;

(10) human parathyroid cancer, from accidental adenoma;

(11) cytoplasm mRNA, from human healthy senile fibroblast which was replaced by healthy epidermis fibroblast and whose phenotype was cell body increased, applanation, cleavage stops (add 5-bromouracil. 48 hours later, the infiltration rate was lower than 2%).

During construction, human fetal brain was constructed with Lamda( ) BA vector, the cDNA libraries of other materials were constructed with pT₇T₃-Pac vector. The cloning site of the fetal liver and spleen cDNA libraries was between enzymatic cleavage sites of PacI and EcoRI, the cloning site of the fetal brain cDNA libraries was between enzymatic cleavage sites of NotI and HindIII, the others were all between enzymatic cleavage sites of NotI and EcoRI.

For example, construction of cDNA libraries from healthy female fetal brain was as follows:

Poly(A)⁺ RNA was extracted from healthy female infant brain (72 days) and used to construct cDNA libraries. Synthesize oligonucleotide 5′-AACTGGAAGAATTCGCGGCCGCAGGAAT₁₈-3′ (SEQ ID NO:1) as the primer to synthesize single-stranded cDNA, the oligonucleotide had a NotI site (underlined). Amplifying by nucleotide capping improves the ratio of full-length cDNA in the products. After electrophoresis, the 2.0 kb cDNA fragments were cut and collected from the gel, then linked with the linker of HindIII. After digestion with NotI, the cDNA was linked with phage L-BA that contained HindIII and NotI sites. Vector L-BA was derived from pEMBL-9(+) (Dente, L., Cesareni, G. & Cortese, R. (1983) Nucleic Acids Res. 11, 1645-1655.). L-BA contained that ampicillin resistance gene, the origin of replication of plasmid and f₁ phage, and a multi-cloning site (5′ HindIII-BamHI-NotI-EcoRI 3′). The recombined phage and the helper phage M13K07(Vieira, J. Messing. 1987. Production of single-stranded plasmid DNA. Methods Enzymol. 153:3-11) were transfected into bacteria, and the double-stranded DNA was converted into annular single-stranded cDNA which comprised the inserted cDNA fragments.

Construction of cDNA libraries of the other materials were the same as the cDNA libraries of healthy female infant brain, except that healthy female infant brain was replaced by other materials; and Lamid BA was replaced by vector pT₇T₃-Pac and NotI/HindIII was replaced by the corresponding restriction endonucleases (PacI/EcoRI or NotI/EcoRI).

Preparation of DNA for Single-stranded cDNA Original Libraries

After purification with reversed phase HPLC and affinity column, the DNA obtained from IB libraries was transformed into DH5α F5′ by electroporation. The strain was incubated in an ampicillin-containing medium at 37° C. until its OD₆₀₀ reached 0.2. The helper phage whose content exceeded 20 times was super-infected. 4 hours later, single-stranded DNA was obtained (Vieira, J. Messing. 1987. Production of single-stranded plasmid DNA. Methods Enzymol. 153:3-11).

In order to decrease the occurrence of RF DNA, the obtained 20 ug DNA was incised with PvuII (PvuII can only incise double-stranded DNA), then extracted with phenol/chloroform, diluted by adding 2 ml loading buffer (0.12M sodium phosphate buffer, pH6.8/10 mM EDTA/1% SDS), then purified at 60° C. with hydroxyapatite chromatography column, the column was pre-equilibrated with the same buffer (1 ml column bed, 0.4 hydroxyapatite). The column was eluted with 6 ml loading buffer, and the solution was combined with fractions passed through the column. The sample was extracted twice with water saturated 2-butanone, once with anhydrous 2-butanone and once with water saturated phenol (3-time extraction solution was added each time). Then the sample was allowed to pass through Nensorb column (DuPont/NEN) to remove the saline ion, precipitated with ethanol, then run on with low melting point agarose gel electrophoresis so as to remove helper phage DNA and tRNA and oligo-ribonucleotide produced by RNase A digestion during purification. The region containing single-stranded libraries DNA on the gel was incised, digested with β-agarase (New English Biolabs). Finally, DNA was purified and precipitated with ethanol.

The above constructed high quality cDNA libraries (IB) from brain and other materials had the following characters: the average length of the inserted fragments in cDNA was 1.7 kb; in general, the genetic information within the encoding region could be obtained by sequencing the 5′ end; the poly(A) tail of mRNA was short; more useful information could be obtained by sequencing the 3′ end; the occurrence of non-recombined clone was very few (0.1%); sequencing more than 2000 clones shown no chimeric cDNA.

Construction of Homogenized cDNA Libraries

It is generally thought that 3′ non-encoding region pairing with its transcript is almost unique, so it can be predicted that 3′ non-encoding region only pairs with its complementary strand. On the contrary, because the encoding region usually represents members of the oligogene or multi-gene family, cross hybridization within the encoding region often results in the decrease of sparse cDNA in libraries during homogenization. The method of the inventors used here can prevent this possibility. At the beginning, single-stranded annular cDNA was used as a template to synthesize a short complementary strand from 3′ terminal and control the length of the strand in a very short range (200±20 nt). Because the average length of the mRNA 3′ non-encoding region was 750 nt (Hawkins, J. D. (1998) Nucleic Acids Res. 16, 9893-9908.), most of the synthesized complementary strand engaged in anneal should lack encoding sequences. However, it was necessary to purify the products by hydroxyapatite chromatography after partial elongation, because when the single-stranded DNA in the IB libraries, which lacked poly(A) tail and could not synthesize primer, passed through the column at the first time, about 0.1% of the DNA would be non-specifically bound onto the column.

The method included the following steps: denaturing, annealing, then purifying the unassociated annular DNA (homogenized libraries) by hydroxyapatite chromatography and transforming into the bacteria by electroporation. In order to synthesize the nucleotide of about 200 nt by restricted elongation, 9 pmol of oligonucleotide primer 5′-GGCCGCAGGAAT₁₅-3′ (SEQ ID NO:2) was added into 4.5 pmol of single-stranded DNA of IB libraries. The volume of the reaction system was 150 ul, containing 30 mM Tris-HCl (pH7.5), 50 mM NaCl, 15 mM MgCl₂, 1 mM DTT, 0.1 mM dNTPs, 2.5 mM ddATP, ddCTP, ddGTP and trace of [α³²P]dCTP. The mixture was placed in water at 60° C. for 5 minutes, then 50° C. for 15 minutes, then cooled down to 37° C.; after adding 75 units of Klenow DNA polymerase (United states Biochemical), placed again in water for 30 minutes. EDTA was added to stop the reaction. The mixture was extracted with phenol/chloroform, then diluted with 2 ml HAP loading buffer containing 50 ug ultrasonicated denatured salmon sperm DNA carrier and passed through hydroxyapatite chromatography column (as mentioned above). Some of the double-stranded DNA bound to the hydroxyapatite was eluted with 6 ml eluting solution (0.4M sodium phosphate buffer, pH6.8/10 mM EDTA/1% SDS). Added 14 ml water containing 50 ug DNA carrier, regulated the concentration of the phosphoric acid to 0.12M, and repeated the above steps. Finally, using the method mentioned above, the obtained eluent was extracted and desalinated, then the DNA was precipitated with ethanol. The precipitate (112 ng) was dissolved in 2.5 ul formamide, added a drop of mineral oil and heated at 80° C. for 3 minutes to denature DNA. 0.5 ul Tris-HCl (pH7.5), 0.1 MEDTA, 0.5 ul 5M NaCl, 1 ul (5 ug) (dT)₂₅₋₃₀, and 0.5 ul (0.5 ug) extending primer were added and the whole volume of the reaction system was adjusted to 5 ul. Subsequently, the anneal reaction was carried on. Finally the two components were used to block the adenine residue (i.e., the original poly(A) tail) and extend the oligonucleotide chain on the single-stranded annular DNA.

The annealed mixture was incubated at 42° C., and after the below mentioned time intervals, 2.5 ul sample was taken out each time:

1 hour later, 2.5 ul sample was taken out (calculated C₀t=0.42); (A₁⁺⁺⁺)

2.36 hours later, 2.5 ul sample was taken out (calculated C₀t=1). (A₂⁺⁺⁺)

The 2.5 ul samples were treated by hydroxyapatite chromatography so as to separate the unbound single-stranded annular DNA from some of the re-bound double-stranded DNA, which were transformed into two highly repeated cDNA homogenized sublibraries (A₁⁺⁺⁺ and A₂⁺⁺⁺), respectively. The single-stranded annular DNA was obtained from the elution peak (as mentioned above). The concentration of the obtained single-stranded annular DNA was measured with spectrophotometer and the above steps were repeated.

The annealed mixture was incubated at 42° C., after the below mentioned time intervals, 1.5 ul sample was taken out each time:

13 hour later, 1.5 ul sample was taken out (calculated C₀t=5.5); (A₁⁺⁺)

23.6 hours later, 1.5 ul sample was taken out (calculated C₀t=10); (A₂⁺⁺)

70.8 hours later, 1.5 ul sample was taken out (calculated C₀t=30); (A₃⁺⁺)

The 2.5 ul samples were treated by hydroxyapatite chromatography so as to separate the unbound single-stranded annular DNA from the partial re-bound double-stranded DNA, which were transformed into three moderately repeated cDNA homogenized sublibraries (A₁^{++.} A₂⁺⁺ and A₃⁺⁺), respectively. The single-stranded annular DNA was obtained from the elution peak (as mentioned above). The concentration of the obtained single-stranded annular DNA was measured with spectrophotometer; and the above steps were repeated.

The annealed mixture was incubated at 42° C., after the below mentioned time intervals, 2.5 ul sample was taken out each time:

118 hour later, 2.5 ul sample was taken out (calculated C₀t=50); (A₁⁺)

188 hours later, 2.5 ul sample was taken out (calculated C₀t=80); (A₂⁺)

The two 2.5 ul samples were treated by hydroxyapatite chromatography so as to separate the unbound single-stranded annular DNA from some of the re-bound double-stranded DNA, which were transformed into two low repeated cDNA homogenized sublibraries (A₁⁺ and A₂⁺), respectively. The single-stranded annular DNA was obtained from the elution peak (as mentioned above) and converted into two rare cDNA homogenized sublibraries (A₁⁻ and A₂⁻). Because the electoporation efficiency of partial double-stranded DNA was 100 times higher than that of the single-stranded DNA (Rubenstein, J. L. R., Brice, A. E. J., Ciaranello, R. D., Denney, D., Porteus, M. H. & Usdin, T. B. (1990) Nucleic Acid Res. 18, 4833-4842.), the random hexamer and T₇DNA polymerase (Sequenase version II; United States Biochemical) were added to extend the primer, and the single-stranded annular DNA was converted into partial double-stranded DNA. The volume of the reaction mixture was 10-20 ul, containing 1 mM dNTPs. EDTA was added to stop the reaction (final concentration was 20 mM). After having been extracted with phenol and precipitated with ethanol, the obtained cDNA was dissolved into 10 mM Tris-HCl, pH7.5/1 mM EDTA, then transformed into competence host (DH10, GIBCO/BRL) by electroporation. In order to measure the number of the transformants, 1 hour after electroporation, 10 ul of bacteria was coated onto a LB plate containing 75 ug/ml ampicillin. The measurement showed that a homogenized library containing 2.5×10⁶ clones was obtained. After extraction with Qiagen plasmid kit (Qiagen, Chatsworth, Calif.), super helix plasmid DNA could be obtained.

Construction of Homogenized and Subtracted cDNA Sublibraries Only Expressed in Healthy Fetal Brain

The homogenizing steps were the same as those mentioned above, except the subtracted homogenizing treatment with synthesized fragments which used cDNA original libraries as template and the constructed human infant brain A₁⁺⁺⁺ and A₂⁺⁺⁺, the cDNA original libraries were: human fetal liver and spleen, human full-term placenta, human 8-9 weeks' placenta, human breast, adult brain, human retina, human conarium, human oophoroma, human melanocyte, human embryonic heart, human parathyroid cancer, human senile fibroblast, human polysclerotic focus, human fetal lung.

The annealed mixture was incubated at 42° C. Then, the samples whose C₀t were 0.42 and 1 respectively were taken to construct two homogenized sublibraries. Thus, the cDNA enriching on the HAP column was the cDNA highly expressed in both of cDNA original libraries, while the elution peak was the abundant cDNA only expressed in A⁺⁺⁺ (A represented human infant brain). These two homogenized sublibraries were designated as: spA₁⁺⁺⁺ and spA₂⁺⁺⁺.

The A⁺⁺ libraries were subtracted and homogenized in the same way. The annealed mixture was incubated at 42° C., while taking the samples whose C₀t were 5.5, 10 and 30, respectively. By collecting the elution peak, three moderate abundance homogenized sublibraries only expressed in A (A represented human infant brain) were obtained, and designated as spA₁⁺⁺,sp A₂⁺⁺ and spA₃⁺⁺.

The A⁺ and A⁻ were subtracted and homogenized in the same way. The annealed mixture was incubated at 42° C., while taking the samples whose C₀t were 50 and 80, respectively. By collecting the elution peak, two low abundance homogenized libraries only expressed in A (A represented human infant brain) were obtained, and designated as spA₁⁺ and spA₂⁺. Further two very low abundance homogenized libraries were obtained, and designated as: spA₁⁻ and spA₂⁻.

For the sublibraries obtained from each step, the large-scale sequencing were carried out by judging whether the cDNA clones were novel; and if so, analyzing the integrity of 5′-terminal until full-length sequence was obtained. All the sequenced long cDNA clones formed the existing cDNA libraries. Then the probe were synthesized, hybridized with the obtained sublibraries and original libraries to eliminate the sequenced cDNA clones and retain unsequenced clones so as to improve the efficiency of the large-scale sequencing. The simple process was shown in FIG. 3. The method of hybridization was as follows.

Colony Hybridization

Duplicate nylon filters (GeneScreenPlus; DuPond/NEN) were treated according to the method of Grunstein, M. & Hogness, D. (1975) Proc. Acad. Sci. USA 72, 3961-3965. The colonies were allow to grow on the membrane so as to screen the libraries. Hybridization was carried on at 42° C. The reaction system contained 50% formamide, 5×Denhardt's solution, 0.75M NaCl, 0.15M Tris-HCl, pH7.5, 0.1M sodium phosphate, 2% SDS, and 100 ug/ml digested and degenerated salmon sperm DNA. Radioactive probes were prepared with synthetic primers and Prime-it II kit (Stratagene)(Feinberg, A. P.& Vogelstein, B. (1983) Anal. Biochem. 132, 6-13. Feinberg, A. P.& Vogelstein, B. (1984) Anal. Biochem. 137, 266-267).

DNA Sequencing

The double-stranded plasmid DNA template was prepared by Wizard Minipreps DNA purification system (Promega), sequenced from both ends with forward and reverse M13 fluorescent primers. The reaction products were measured by Biomek 1000 workstation (Beckman). The products were then transferred to a PCR amplifier (Perkin-Elmer/Cetus) to conduct cycling measurement. The reaction products were analyzed by 370 DNA automatic sequencer (Applied Bios). The research of nucleic acid and protein databases was carried out using BLAST algorithm (Altschul, S. F., W. Niller, E. Myers, and D. J. Lipman. 1990. Basic local alignment search tool. J.Mol.Biol. 215:403-410.) on the server of the National Center for Biotechnology Information.

### Example 2

Construction of Homogenized and Subtracted cDNA Sublibraries Only or Mainly Expressed in Infant (72 days) Brains of Spinomyotrophic Syndrome and the Large Scale Sequencing

Construction of High Quality cDNA Original Libraries from Different Tissues or Cells

According to the manufacturer's instruction, Oligotex mRNA kit was used to purify poly(A)+ RNA from infant (72 days) brains of spinomyotrophic syndrome and other cells, except the senile fibroblast used to separate cytoplast RNA. Twice purification was needed. The construction of cDNA libraries was very important. In typical reactions, 1 ug of poly(A)+RNA and 2 times of oligo(dT)₁₈-Not I primer (PacI primer was used for liver/spleen libraries) were annealed at 37° C. and then reverse transcribed at 37° C. with reverse transcriptase (Life Technologies). Poly(A)+RNA also could be annealed by adding 4 times of (dT)₂₅-Not I primer at 45° C. and then reverse transcribed at 45° C. Tag is a sequence of 2-6 nucleotides which was specific for the different libraries. So it could be used to identify libraries. Except fetal brain, fetal liver/spleen, and placenta libraries, the first strand of cDNA of all the other libraries was synthesized using the following oligonucleotide primer: TGTTACCAATCTGAAGTGGG AGCGGCCGC-tag-(dT)₁₈or₂₅. (SEQ ID NOS:3 and 4). The first strand of cDNA of fetal heart, brain and placenta libraries was synthesized using the following primer: AACTGGAAG AATTAATTAAAG-ATCT(dT)₁₈ (Pharmacia) (SEQ ID NO:5). This primer was used in the synthesis of the first strand of cDNA of embryonic liver/spleen. Double-stranded cDNA was filtered and separated by polyacrylamide gel column (length 64 cm, diameter 0.2 cm) and then ligated with 500 to 1000 times linkers. Infant brain cDNA was ligated by Hind III linker and cut by Not I, separated by polyacrylamide gel column again and finally cloned to the site between NotI and Hind III of vector Lafmid BA (Soares, M. B.1994. Construction of directionally cloned cDNA libraries in phagemid vectors. In Automated DNA sequencing and analysis (ed. M. D. Adams, C. Fields and J. C. Venter), pp.110-114. Academic Press, New York, N.Y.USA 74,1020-1023.). Embryonic liver/spleen cDNA libraries were ligated by EcoRI linker and cut by PacI and then cloned between PacI and EcoRI of vector pT₇T₃-Pac (Pharmacia). pT₇T₃-Pac was a modified polylinker like pT₇T₃-Pac 18D and could transform E.coli and construct cDNA original libraries from different tissues or cells. Plasmid DNA from the cDNA original libraries was purified on HPLC reverse-phase column and affinity column, transformed via electroporation into DH5αF′. The following method was used get different single-stranded and double-stranded high quality cDNA original libraries.

The following was the sequence of the pT₇T₃-Pac polylinker (SEQ ID NO:6):

| | |
|---|---|
| 5′-CACCCCAGGC TTTACACTTT ATGCTTCCGG CTCGTATGTT GTGTGGAATT | 50 |
| GTGAGCGGAT AACAATTTCA CACAGGAAAC AGCTATGACA TGATTACGAA | 100 |
| M13 reverse sequencing primer | |
| TTTAATACGA CTCACTATAG GGAATTTGGC CCTCGAGGCC AAGAATTCCC | 150 |
| T7 promoter SfiI EcoRI | |
| GACTACGTAG TCGGGGATCC GTCTTAATTA AGCGGCCGCA AGCTTATTCC | 200 |
| SnaBI BamHI PacI NotI HindIII | |
| CTTTAGTGAG GGTTAATTTT AGCTTGGCAC TGGCCGTCGT TTTACAACGT | 250 |
| T3 promoter | |
| CGTGACTGGG AAAACCCTGG CGTTACCCAA CTTAATCGCC TTGCAG-3′ | 296 |
| M13 sequencing primer | |

Construction of Purified and High Quality Single-stranded Annular DNA Original Libraries in vitro

According to the manufacturer's instruction (Life Technologies,Catalogue10356-020), use Gene II (phagemid F1 endonuclease) and E.coli exonuclease III (Exo III) to convert phage's double-stranded DNA into single-stranded DNA. As described above (Soares, M. B.1994. Construction of directionally cloned cDNA libraries in phagemid vectors. In Automated DNA sequencing and analysis (ed. M. D. Adams, C. Fields and J. C. Venter), pp.110-114. Academic Press, New York, N.Y.USA 74,1020-1023.), the derived single-stranded DNA can be purified by HAP chromatography (Bio-Rad) from residual double-stranded plasmid DNA. The original protein of phage F1 replication is a kind of specific endonuclease that can bind to phage F1 start site and make a cut in the superhelix DNA. The strand having the cut can be cleaved later by Exo III and form single-stranded loop (Hoheisel, J. D. 1993. On the activities of Escherichia coli exonuclease III. Anal.Biochem. 209:238-246.). Gene II's function of loosing superhelix plasmid is not completed so HAP is needed to purify single-stranded loop. GeneII reacted for 1 h at 37° C. and a typical reaction includes 4 ul superhelix plasmid of cDNA libraries, 1 ul GeneII (Life Technologies), 2 ul 10×GeneII buffer (Life Technologies), totally 20 ul. Genell was deactivated at 65° C. for 5 min, cooled on ice and then 2 ul Exo III was added, incubated for 30 min at 37° C., GeneII and Exo III (Life Technologies, Cataloguel18013-011,65 u/ul) were cut by protease K (Boehringer Mannheim) at 50° C. for 15 min. The total volume was 100 ul, including 10 mM Tris (PH7.8), 5 mM EDTA, 0.5% SDS and 136 ul protease K. Then it was eluted with phenol, chloroform and isoamyl alcohol (25:24:1) of the same volume, precipitated with ethanol, reacted with PvuII at 37° C. for 2 h and converted the residual superhelix plasmid into linearized DNA to increase the affinity with HAP. PuvII could not cut single strand, so there were two PuvII sites in vector. The reacted mixture was diluted by adding 2 ul buffer [0.12M sodium phosphate (pH6.8), 10 mM EDTA and 1% SDS] and purified with HAP affinity chromatography pre-equilibrated by the same buffer (bed volume is 1 ml or 0.4 g HAP) at 60° C. The mixture was washed with 6 ml buffer and combined the washed buffer with the flow-though, extracted with saturated 2-butanol 2 times, anhydrous 2-butanol once and saturated ether once (Three times of the volume were used each time). Residual ether was dried by vacuum, and desalinated on a Nensorb column (DuPont/NEN) according to manufacturer's instruction and finally concentrated to 0.35 ml and precipitated with ethanol. It should be pointed out that the polarity of single-stranded DNA made by GeneII and Exo III is opposite to that of the single-stranded DNA produced in the phagemid.

Production of High Quality Covalently Closed Single-stranded cDNA Original Libraries in vivo

According to the method described in the reference (Vieira, J.& Messing, J.(1978) Methods Enzymol. 153,3-11.), plasmid DNA from original libraries was transformed into E.coli DH5αF′ by electroporation, and cultured to OD₆₀₀ under the selection of ampicillin at 37° C. and super-infected by 10-20 times of helper phage M13KO₇ (Pharmacia). Single-stranded annular plasmid was harvested after 4 hours of cultivation.

Converting the Single-stranded Loop Into the Double-stranded Plasmid

Single-stranded loop was precipitated by ethanol (<50 ug) and resuspended in 11 ul water. After adding 4 ul 5×sequencing enzyme buffer (USB) and 1 ul primer (1 ug), the mixture was incubated at 65° C. for 5 min. at 37° C. for 3 min. The mixture was then incubated at 37° C. for 30 min before adding 1 ul sequencing enzyme (UBS 2.0), 1 ul 0.1M DTT, 2 ul mixed dNTP (The final concentration of each dNTP was 10 mM). The mixture was extracted by phenol, chloroform and isoamyl alcohol (25:24:1). Added 10 mM Tris(PH8.0) and 1 mM EDTA (TE) until the total volume was 100 ml.

Plasmid DNA was precipitated by ethanol and dissolved into 3 ul TE. Oligonucleotides as follows were used in the reaction of primer elongation:

(1) M13 reverse sequencing primer (5′-AGCGGATAACAATTTCACACAGGA-3′) (SEQ ID NO:7), which was complemented to the single strand outside the bodies.

(2) Oligomeric Amp (5′-GACTGGTGAGTACTCAACCAAGTC-340 )(SEQ ID NO:8), which was complementary to single-stranded pT₇T₃-Pac from bodies or single-stranded Lafmid BA plasmid of anti-ampicillin gene.

These high quality cDNA libraries established as described had the following characteristics: the average length of inserted fragments was 1.7 kb; the genetic information of coding regions usually could be obtained by sequencing the 5′ end; the poly(A) tail of mRNA was short; more useful information could be obtained by sequencing the 3′ end; the occurrence of non-recombined clones was very low (0.01%); sequencing more than 2000 clones has revealed no chimeric cDNA.

Construction of cDNA Homogenized Sublibraries

This is a method based on renaturation dynamics. This method is to hybridize excessive 20 times inserted cDNA fragments produced from PCR with single-stranded libraries, purify the residual single-stranded plasmid with HAP and convert them into double strand, transform them into DH10B by electroporation and finally select them by ampicillin. According to the instruction, PCR amplification of inserted cDNA fragment was carried out in an amplification system of high fidelity (Boehringer Mannheim) which contains a mixture of Taq and Pwo DNA enzymes (Barnes, W. M.1994. PCR amplification of up to 35-kb DNA with high fidelity and high yield from lambda bacteriophage templates. Proc.Natl.Acad.Sci. 91:2216-2220.). 1 ul (2.5-5.0) DNA template [double-stranded plasmid (cDNA original libraries of infant brains of spinomyotrophic syndrome, embryonic lung, hypoparathyroid adenoma, senile fibroblast) or single-stranded annular DNA (cDNA original libraries of embryonic heart, embryonic liver/spleen) prepared ex vivo] and 2 ul reserved dNTP solution (final concentration of each dNTP was 200 m in reaction), 5 ul 20 uM T₇ primer (540 -TAATACGACTCACTATAGGG-3′) (SEQ ID NO:9), 5 ul 20 uM T₃ primer (5′-ATTAACCCTCACTAAAGGGA-3′)(SEQ ID NO:10), 10 ul 10×buffer of the high fidelity amplification system, 0.75 ul enzyme (2.6 u) of the high fidelity amplification system, 76.25 ul water and 50 ul mineral oil were added. The reaction was carried out in a Perkin Elmer cycler. The reaction was performed under the following conditions: first rise from room temperature to 94° C. for 7 min, incubate at 9420 C. for 1 min, at 55° C. for 2 min, at 72° C. for 3 min, at 72° C. for 7 min. and 20 cycles. Fragment PCR were purified by high purity PCR product purification kit, and then dissolved into 5 ul TE. 1.5 ul (0.5 ug) was taken and mixed with 5 ul (50 ug) DNA (single-stranded annular DNA produced in vitro), deionized formamide, 0.5 ul (10 ug) 5′ blocking oligomer AV-1 (5′-CCTCGTGCCGAATTCTTGGCCTCGAGGGCCAAATTCCCTATAGTGAGTGTATTA-3′) (SEQ ID NO:11), 0.5 ul (10 ug) 3′ blocking oligomer AR (5′-ATTAACCCTCACTAAAGGGAATAAGCTTGCGGCCGCT₂₀-3′(SEQ ID NO:12), which was used for all the libraries except embryonic liver/spleen) or 0.5 ul(10 ug)3′ AV-2 (5′-ATTAACCCTCACTAAAGGGAATAAGCTTGCGGCCGCTTAATTAAAGATCT₁₉-3′ (SEQ ID NO:13) only used for embryonic liver/spleen), and sealed with 10 ul mineral oil. The mixture was heated at 80° C. for 3 min. Added 1 ul 10×buffer-A [1.2M NaCl, 0.1M Tris(PH8.0) and 50 mM EDTA, for cDNA original libraries of infant brains of spinomyotrophic syndrome, embryonic lung, embryonic heart, hypoparathyroid adenoma, senile fibroblast] or add 1 ul buffer-B [1.2M NaCl, 0.1M Tris(PH8.0), 50 mM EDTA and 10% SDS, used for cDNA original libraries of 14Nb2HFL20W-embryonic liver/spleen] and 1.5 ul water. Hybridization was performed at 30° C. and the following time or calculated C₀t. Residual single-stranded DNA was purified by HAP, converted into double strand, and transformed into DH10B by electroporation.

The following example is used to explain the construction of the homogenized cDNA sublibraries and B denotes cDNA original libraries of infant brains of spinomyotrophic syndrome. The annealed mixture described above was incubated at 30° C. and 2.5 ul of samples were taken at following time intervals:

Take 2.5 ul sample after 0.17 hour(calculated C₀t=0.42), (B₁⁺⁺⁺)

Take 2.5 ul sample after 0.4 hour(calculated C₀t=1), (B₂⁺⁺⁺)

The 2.5 ul samples were treated by hydroxyapatite chromatography so as to separate the non-hybridized single-stranded annular DNA from some of the re-bound double-stranded DNA, which were transformed into two highly repeated cDNA homogenized sublibraries (B₁⁺⁺⁺ and B₂⁺⁺⁺), respectively. The single-stranded annular DNA was obtained from the elution peak (as mentioned above). The concentration of the obtained single-stranded annular DNA was measured with spectrophotometer and the above steps were repeated.

The annealed mixture was incubated at 42° C. and 1.5 ul of sample was taken at following time intervals:

Take 1.5 ul sample after 2.2 h.(calculated C₀t=5.5), (B₁⁺⁺)

Take 1.5 ul sample after 4 h.(calculated C₀t=10), (B₂⁺⁺)

Take 1.5 ul sample after 12 h.(calculated C₀t=30),(B₃⁺⁺)

The 1.5 ul samples were treated by hydroxyapatite chromatography so as to separate the non-hybridized single-stranded annular DNA from some of the re-bound double-stranded DNA, which were transformed into three moderately repeated cDNA homogenized sublibraries (B₁⁺⁺, B₂⁺⁺ and B₃⁺⁺), respectively. The single-stranded annular DNA was obtained from the elution peak (as mentioned above). The concentration of the obtained single-stranded annular DNA was measured with spectrophotometer and the above steps were repeated.

The annealed mixture was incubated at 42° C. and 2.5 ul of sample was taken at following time intervals:

Take 2.5 ul sample after 20 .h. (calculated C₀t=50), (B₁⁺)

Take 2.5 ul sample after 32 .h. (calculated C₀t=80), (B₂⁺)

The two 2.5 ul samples were treated by hydroxyapatite chromatography so as to separate the non-hybridized single-stranded annular DNA from some of the re-bound double-stranded DNA, which were transformed into two low repeated cDNA homogenized sublibraries (B₁⁺ and B₂⁺), respectively. The single-stranded annular DNA was obtained from the elution peak (as mentioned above) and converted into two rare cDNA homogenized sublibraries (B₁⁻ and B₂⁻). Because the electoporation efficiency of partial double-stranded DNA was 100 times higher than that of the single-stranded DNA (Rubenstein, J. L. R., Brice, A. E. J., Ciaranello, R. D., Denney, D., Porteus, M. H. & Usdin, T. B. (1990) Nucleic Acid Res. 18, 4833-4842.), the random hexamer and T₇DNA polymerase (Sequenase version II; United States Biochemical) were added to extend the primer, and the single-stranded annular DNA was converted into partial double-stranded DNA. The volume of the reaction mixture was 10-20 ul, containing 1 mM dNTPs. EDTA was added to stop the reaction (final concentration was 20 mM). After having been extracted with phenol and precipitated with ethanol, the obtained cDNA was dissolved into 10 mM Tris-HCl, pH7.5/1 mM EDTA, then transformed into competence host (DH10, GIBCO/BRL) by electroporation. In order to measure the number of the transformants, 1 hour after electroporation, 10 ul of bacteria was coated onto a LB plate containing 75 ug/ml ampicillin. The measurement showed that a homogenized library containing 2.5×10⁶ clones was obtained. After extraction with Qiagen plasmid kit(Qiagen, Chatsworth, Calif.), super helix plasmid DNA could be obtained.

Constitution of Homogenized cDNA Libraries Expressed Only or Mainly in Infant Brain Cells of Spinal Atrophia

The following is the subtraction of the constituted homogenized cDNA libraries for infant brain cells of spinal atrophia. According to the description, double-stranded cDNA plasmid may be prepared using Olagen Midi-prep kit. These DNAs come from high-class cDNA original libraries of human fetal brain, adult brain, human fetal liver and spleen, human full-term placenta, human 8-9 weeks placenta, human breast, human retina, human pineal gland, human oophoroma, human melanocyte, human fetal heart, human parathyroid cancer, human senility fibroblast, human multiple scleroderma focus, human fetal lung, etc. Then double-stranded plasmid DNA is converted into single strand in vitro. As mentioned above, single-stranded cyclic DNA was purified by HAP chromatography and used as the template of PCR amplification with primer T7 and T3. The single-stranded cyclic DNA from constituted homogenized cDNA libraries (in 2 ul deionized formamide) for fetal brain cells of spinal atrophia was blended with the amplified DNA inserted fragments obtained from the whole 1.5 ug cDNA original libraries (in 4 ul deionized formamide). After adding 2.1 ul (42 ug) of 5blocking oligomer AV-1 and 2.1 ul (42 ug) 3 blocking oligomer AV-2, 10 ul mineral oil was added. Heated 30 minute at 80° C., and then added 1.2 ul of 10×buffer B and 0.6 ul of water. The hybridization was carried out at 30° C. and the following time or calculated C₀t. The non-hybridized single-stranded DNA was purified by HAP, converted into double strand, and transformed into DH10B by electroporation. After selection with ampicillin, the subtracted and homogenized cDNA libraries only or mainly expressed in fetal brain cells of spinal atrophia were obtained. The DNAs bound with HAP were treated and purified in the same way in order to control the experiment.

The above DNAs were annealing with B1⁺⁺⁺ and B2⁺⁺⁺ at 30° C. for 0.73 hour and 1.8 hours, respectively. The corresponding C₀t values were 0.42 and 1, respectively. In this way, cDNAs enriched in HAP were those highly expressed in all cDNA original libraries. Those in elution peaks were cDNAs that were only highly expressed in B⁺⁺⁺. These libraries were designated as spB1⁺⁺⁺ and spB2⁺⁺⁺.

The B⁺⁺ libraries were subtracted and homogenized in the same way. The annealed mixture was incubated at 30° C. for 9.7 h, 17.7 h and 53.3 h, which corresponded to C₀t values of 5.5, 10 and 30, respectively. By collecting the elution peak, three moderate abundance homogenized sublibraries only expressed in B were obtained, and designated as spB₁⁺⁺,sp B₂⁺⁺ and spB₃⁺⁺.

The B⁺ and B⁻ were subtracted and homogenized in the same way. The annealed mixture was incubated at 30° C. for 88.9 h and 142 h, which corresponded to C₀t values of 50 and 80, respectively. By collecting the elution peak, two low abundance homogenized libraries only expressed in B were obtained, and designated as spBhd 1⁺ and spB₂⁺. Further two very low abundance homogenized libraries were obtained and designated as: spB₁⁻ and spB₂⁻.

For the sublibraries obtained from each step, the large-scale sequencing were carried out by judging whether the cDNA clones were novel; and if so, analyzing the integrity of 5′-terminal until full-length sequence was obtained. All the sequenced long cDNA clones formed the existing cDNA libraries. Then the probe were synthesized, hybridized with the obtained sublibraries and original libraries to eliminate the sequenced cDNA clones and retain unsequenced clones so as to improve the efficiency of the large-scale sequencing. The simple process was shown in FIG. 3. The method of hybridization was as follows.

Colony Hybridization

The duplicate nylon filters (GeneScreenPlus; DuPond/NEN) were treated according to the method of Grunstein, M. & Hogness, D. (1975) Proc. Acad. Sci. USA 72, 3961-3965. The colonies were allow to grow on the membrane so as to screen the libraries. Hybridization was carried on at 42° C. The reaction system contained 50% formamide, 5×Denhardt's solution, 0.75M NaCl, 0.15M Tris-HCl, pH7.5, 0.1M sodium phosphate, 2% SDS, and 100 ug/ml digested and degenerated salmon sperm DNA. Radioactive probes were prepared with synthetic primers and Prime-it II kit (Stratagene)(Feinberg, A. P.& Vogelstein, B. (1983) Anal. Biochem. 132, 6-13. Feinberg, A. P.& Vogelstein, B. (1984) Anal. Biochem. 137, 266-267).

DNA Sequencing

The double-stranded plasmid DNA template was prepared by Wizard Minipreps DNA purification system (Promega), sequenced from both terminals with forward and backward M13 fluorescent primers. The reaction products were measured by Biomek 1000 workstation (Beckman). Then the products were transferred to PCR amplifier (Perkin-Elmer/Cetus) to conduct cycling measurement. The reaction products were analyzed by 370 DNA automatic sequencer (Applied Bios). The research of nucleic acid and protein databases was carried out using BLAST algorithm (Altschul, S. F., W. Niller, E. Myers, and D. J. Lipman. 1990. Basic local alignment search tool. J.Mol.Biol. 215:403-410.) on the server of the National Center for Biotechnology Information.

The efficiency of obtaining new sequences was well over 10% through 50 cycles of sequencing and subtractive hybridization.

All the documents cited herein are incorporated herein in their entirety by reference. Further, it is appreciated that, in the above teaching of the invention, one of ordinary skill in the art can make certain changes or modifications to the invention, and these equivalents are still within the scope of the invention defined by the appended claims of the present application.

We claim:

1. A method of cDNA sequencing comprising:
(1) constructing at least one cDNA original library using ribonucleic acid isolated from cells of an organism in which the length of inserted fragments is 0.5-3.0 kb;
(2) homogenizing the cDNA original library according to a graded C₀t value, wherein C₀ is concentration of total DNA (in mol/L) based on the number of nucleic acids; and t is the renaturing time (in seconds);
(3) selecting and sequencing 5-500 clones from the homogenized cDNA library;
(4) synthesizing one or more probes corresponding to clones sequenced in (3), and hybridizing and subtracting the homogenized cDNA library with said probes; and
(5) repeating (3) and (4) 1-5,000 times.

2. The method of claim 1, wherein the ribonucleic acid is isolated from cells selected from the group consisting of cells at a selected stage of growth, development, or circadian oscillation, cells that display pathological features, and cells that comprise a particular tissue.

3. The method of claim 2, wherein more than one cDNA original library is constructed.

4. The method of claim 3 further comprising,
homogenizing said cDNA original libraries respectively to obtain homogenized libraries of different tissues; and
hybridizing and subtracting among said homogenized cDNA libraries of different tissues.

5. The method of claim 3, wherein the cells used to prepare one cDNA original library differ from the cells used to prepare another cDNA original library(ies) in stage of growth, development, or circadian oscillation, pathological features, and/or tissue of origin.

6. The method of claim 1, wherein (4) further comprises:
synthesizing probes based on known cDNA sequences, or synthesizing probes based on other sequenced clones; and hybridizing and subtracting the homogenized cDNA libraries of the preceding step with said probes.

7. The method of claim 1, wherein (3) further comprises:
determining whether sequenced fragments are new cDNA clones;

8. The method of claim 7, wherein (3) further comprises:
analyzing the integrity of 5′ end of new cDNA clones;

9. The method of claim 8, wherein (3) further comprises:
sequencing clones that have an intact 5′ end until obtaining the full-length sequences.

10. The method of claim 1, wherein there are from 3 to 8 grades of C₀t.

11. The method of claim 10, wherein C₀t is divided into 3 grades: 0<C₀t<1, C₀t=1-50, and C₀t>50.

12. The method of claim 1, wherein constructing cDNA original libraries comprises:
extracting mRNA, amplifying mRNA to obtain the corresponding cDNA by a technique selected from the group consisting of: a mixed reverse transcriptase technique, a SMART PCR technique, a nucleotide capping technique and combinations thereof;
separating and collecting cDNA fragments of 0.5-3.0 kb;
cloning the separated cDNA fragments into suitable vectors;
separating the vectors comprising the inserted fragments of 0.5-3.0 kb; and
transforming into suitable bacteria.

13. The method of claim 1, wherein constructing cDNA original libraries comprises:
extracting mRNA, amplifying the mRNA to obtain the corresponding cDNA by a technique selected from the group consisting of: a mixed reverse transcriptase technique, a SMART PCR technique, a nucleotide capping technique and combinations thereof;
separating and collecting cDNA fragments of 0.5-3.0 kb;
cloning the separated cDNA fragments into suitable vectors;
separating the vectors comprising the inserted fragments of 0.5-3.0 kb;
transforming separated vectors into suitable bacteria; and
extracting DNA from the cDNA libraries, passing the DNA through a poly(T)₁₀₋₂₅ affinity chromatography column, collecting the cDNA bound with poly(T)₁₀₋₂₅ and transferring said cDNA into suitable bacteria.

14. The method of claim 10 further comprising separation and collection of cDNA fragments of 0.5-3.0 kb by electrophoresis and gel excision or by gel chromatography purification; and separation of the vectors comprising the inserted fragments of 0.5-3.0 kb by reversed phase HPLC.

15. The method of claims 11, further comprising separation and collection of cDNA fragments of 0.5-3.0 kb by electrophoresis and gel excision or by gel chromatography purification; and separation of the vectors comprising the inserted fragments of 0.5-3.0 kb by reversed phase HPLC.
